(19) United States
(12) Patent Application Publication
(10) Pub. No.: US 2026/0069136 A1
(43) Pub. Date: Mar. 12, 2026

(54) DEVICE FOR MEASURING THE LENGTH OF AN OBJECT

(71) Applicant: Heidelberg Engineering GmbH, Heidelberg (DE)

(72) Inventors: Andreas FRITZ, Lübeck (DE); Gerit DRÖGE, Lübeck (DE); Ralf ENGELHARDT, Lübeck (DE); Björn MARTENSEN, Lübeck (DE)

(73) Assignee: Heidelberg Engineering GmbH, Heidelberg (DE)

(21) Appl. No.: 19/106,286

(22) PCT Filed: Jun. 22, 2023
(86) PCT No.: PCT/EP2023/066957

§ 371 Date: Feb. 25, 2025

(30) Foreign Application Priority Data
Aug. 25, 2022 (DE) 10 2022 003 104.3
Sep. 14, 2022 (DE) 10 2022 123 426.6

(51) Int. Cl. A61B 3/10; A61B 3/00

(52) U.S. Cl. CPC A61B 3/1005; A61B 3/0058; A61B 3/102; A61B 2560/0238

(57) ABSTRACT

In view of the problem of making the intrinsically competing objects of high resolution or image quality and generation of the smallest possible data rates when measuring object lengths as compatible as possible, a device (1) for determining the length of an object (6) when carrying out optical coherence tomography comprising an interferometer with a light source (2), a sample arm (4) and a reference arm (5), wherein the light emitted by the light source (2) is splitable by a beam splitter (3) such that first light (4a) is guidable in an outward and return direction on the sample arm (4) and second light (5a) is guidable in an outward and return direction on the reference arm (5), wherein the first and the second returning light (4a, 5a) can be made to interfere, wherein an evaluation unit (8) for acquiring and processing signals from the interfering first and second light (4a, 5a) is arranged and wherein a path length switching unit (13a, 13b) is arranged in the beam path of the sample arm (4) and/or in the beam path of the reference arm (5) and modifies the optical path length of the respective light (4a, 5a) passing through the path length switching unit (13a, 13b), is characterized in that at least one path length switching unit (13a, 13b) changes an optical path length from a first value to a second value alternately in time.

The invention relates to a device according to the preamble of claim 1.

The term “optical coherence tomography” (usually abbreviated by OCT) is understood to be an imaging method.

With this method, two-dimensional and three-dimensional images can be obtained from light-scattering structures. In this method, light with a specific bandwidth is typically split into two partial beams in a beam splitter. The first partial beam is incident on the sample or object to be examined, the second partial beam passes through a reference section.

The light reflected by the sample or object interferes with the reference beam. Signals from the interference can be used to examine the sample with a depth resolution, i.e. in the depth of the optical axis of the first partial beam, by means of what are known as A-scans.

In addition, it is possible to also scan the surface of the sample or to scan the sample laterally with the first partial beam in order to obtain OCT images.

Against this background, WO 2012/104 097 A1 has disclosed a method for recording slice images in which a path length switching unit is used. The path length switching unit modifies the path length of a sample beam and/or of a reference beam of an interferometer such that depth slice images can be created at different depths of a sample. The path length is modified by diverting beam paths over different geometric paths.

Currently, a correspondingly large measuring depth of approx. 40-45 mm is required to be able to measure the eye length, i.e. the length from the cornea to the retina, with full OCT resolution, i.e. with a resolution of less than 10 μm. In this case, the whole depth is measured at full resolution. Another approach may consist in reducing the resolution, i.e. the OCT bandwidth.

Very high data rates arise when the full OCT bandwidth or resolution is used; these data rates cannot be dealt with or processed or can only be dealt with or processed with difficulties. The technical outlay and the associated costs are correspondingly high. However, reducing the resolution would be accompanied by the disadvantage that the measurement result quality suffers, and the image quality is perceivably reduced.

Therefore, the problem addressed by the present invention is that of reconciling, to the best possible extent, the actually competing objects of obtaining a high resolution or image quality and creating the lowest possible data rates when measuring object lengths.

The present invention solves the aforementioned problem by way of the features of claim 1.

According to the invention, at least one path length switching unit is used to change an optical path length from a first value to a second value alternately in time in order to detect substantially only two structures that are required for measuring the length of an object, for example a human eye.

At first, it was recognized that the approaches mentioned at the outset are disadvantageous to the effect that large portions of the data records acquired during the measurement of a human eye would also cover the relatively uninteresting region of the vitreous humor, which generally does not provide any information of relevance to the eye length measurement.

Against this background, it was further recognized that the teaching of WO 2012/104 097 A1 requires an extension in order, as it were, to mask the relatively uninteresting region of the vitreous humor. According to the invention, the path length switching unit described in the aforementioned document is used such that there is a back-and-forth alternation between imaging of the corneal region and imaging of the retinal region. In this case, the intrinsic image depth of the OCT system can be substantially smaller than the length of the eye.

This invention allows the eye length to be measured with a very high precision of approx. 10 μm for the first time, wherein the measurement speed, the signal-to-noise ratio (SNR) and the image quality are optimized at the same time and wherein the data rates are simultaneously so low that they mirror the requirements of an OCT with a comparatively small image depth (approx. 10 mm).

The path length switching unit could change the first value of the optical path length to the second value after a defined time interval and subsequently, following the expiry of the time interval or a further time interval, change the second value back to the first value. This allows an automated path length variation which enables reproducible measurements.

The path length switching unit could repeat the alternating change of values over a predeterminable time period, preferably in periodic fashion at a defined frequency. Predetermining the time period as measurement time period and/or predetermining the frequency renders the signal-to-noise ratio variably adjustable and optimizable.

Against this background, the frequency could lie in the range of 1 to 1000 Hz. Data rates that are processible without problems arise in this frequency range.

The difference between the optical path lengths at the first value and at the second value could be variably adjustable on the basis of the length of the examined object to be measured, wherein the expected length can be input into the evaluation unit or into a control unit as an external parameter. This allows a device calibration.

Structures of the object could be ascertainable by the evaluation unit from the acquired and processed signals, the structures being in each case detectable while the first value and the second value are set, with the spatial distance between these structures being ascertainable and able to be output by the evaluation unit as the length of the object. In the example of a human eye, this allows detection of the cornea and the retina as structures and use of their distance to ascertain the length of the eye.

The path length offset between the two paths or path lengths is preferably chosen such that all relevant eye lengths are covered. The offset is calibrated precisely when the device is put into operation. Fast alternation between the two positions and the subsequent detection of the positions of cornea and retina in the OCT signals allows the overall eye length to be inferred if the offset is known.

Provision could be made for an adjustable telescope that serves to keep in focus a structure to be captured. This can improve the signal-to-noise ratio (SNR) in the region of the structure. Specifically, the imaging in two imaging paths could be adapted by individual optics such that the corneal imaging has a focus in the region of the cornea. The retinal imaging is designed such that there is a focus in the region of the retina. This ensures an optimal signal-to-noise ratio (SNR) in both regions. An adjustable focus telescope that compensates for a potential refractive error of the eye so as to always keep the retina in focus could be used to further optimize the SNR.

The evaluation unit could differentiate the conjugate complex plane of a signal from the real plane of the signal. This allows data rates to be reduced. To reduce the data rates, it would be possible, in particular but not exclusively, to differentiate the conjugate complex (CC) plane of the signal from the real plane of the signal in the region of the retinal scan—in a manner similar to full-range OCT.

Against this background, a respective numerical phase correction could be performable using the evaluation unit in order to ascertain the plane in which a signal of a structure of the object to be captured is located. A numerical phase correction could be used to ascertain the plane—complex or real—in which the signal of the retina is located, and hence ascertain the real distance of said signal of the retina from the reference arm. This is possible by applying a respective real phase correction and a respective complex conjugate phase correction to each measurement and by comparing the two signals to one another. Hermitian symmetry for FD-OCT, which makes it difficult to ascertain unique optical lengths between two image regions, can be broken thereby.

A dispersive element, preferably a light-guiding fiber, could be arranged in the reference arm or in the sample arm. If a sufficiently strong dispersion is introduced into the OCT interferometer or OCT setup, then there is a significant amplitude difference following a Fourier transform of the signals since signals not originating from the plane fitting to a phase vector are strongly distorted. If the interferometer itself exhibits hardly any dispersive properties, then optical elements with dispersive properties can be inserted such that this effect becomes sufficiently large. For instance, this can be implemented by way of a piece of fiber with dispersive properties in the reference arm or sample arm.

A device of the type described herein could be used in a method for determining the length of an eye, with at least one path length switching unit changing an optical path length from a first value to a second value alternately in time in order to bring or focus light into the region of the cornea at the first value and in order to bring or focus light into the region of the retina at the second value. The length of an eye can be reliably ascertained thereby.

Against this background, the local position of the cornea could be detected at the first value and the local position of the retina could be detected at the second value, with the length of the eye being ascertained from the distance of the positions from one another.

The movement of the eye during the measurements could be detected by evaluating a plurality of measurements, or a series of measurements, of the positions of cornea and retina, with the results of this evaluation being used to correct errors when detecting the cornea and the retina. This can compensate for movements of the eye in order to increase the measurement accuracy when determining the length of the eye.

The retina or its associated signal could be situated either in a real plane or a real image component of an OCT image or in a complex conjugate plane or a complex conjugate image component of an OCT image, with either the movement trajectories of cornea and retina being evaluated for determining the length of the eye or with a phase analysis of the signals from the cornea and retina being performed.

Images of the cornea and the retina could be displayed and/or displayed on a monitor in real time. This allows a person to assess and evaluate the images.

The device described herein can carry out all method steps described herein, either individually or in combination.

The device described herein can be used for eye length measurement, axis length measurement, biometry and fundus length measurement.

In the drawing:

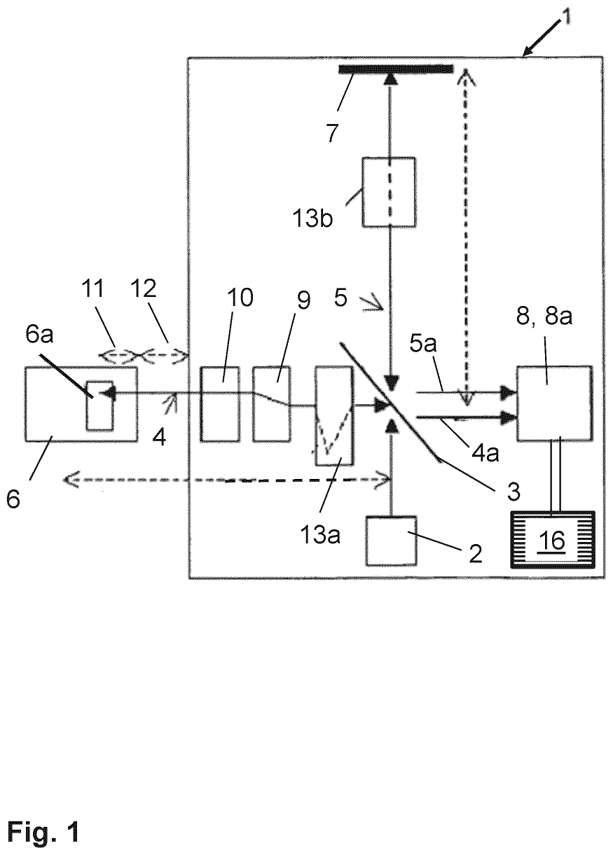

FIG. 1 shows a schematic illustration of a device having a path length switching unit,

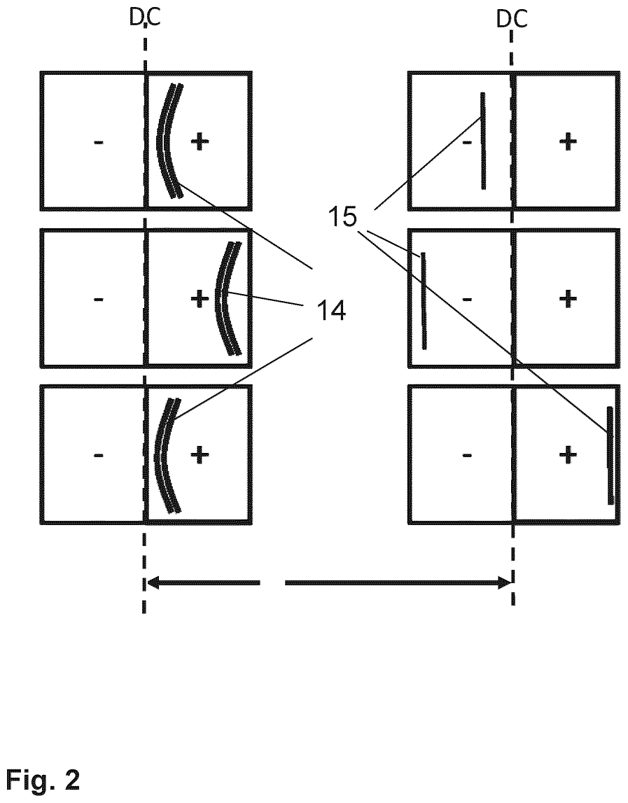

FIG. 2 shows a schematic illustration of imaging in the corneal region and in the retinal region, and

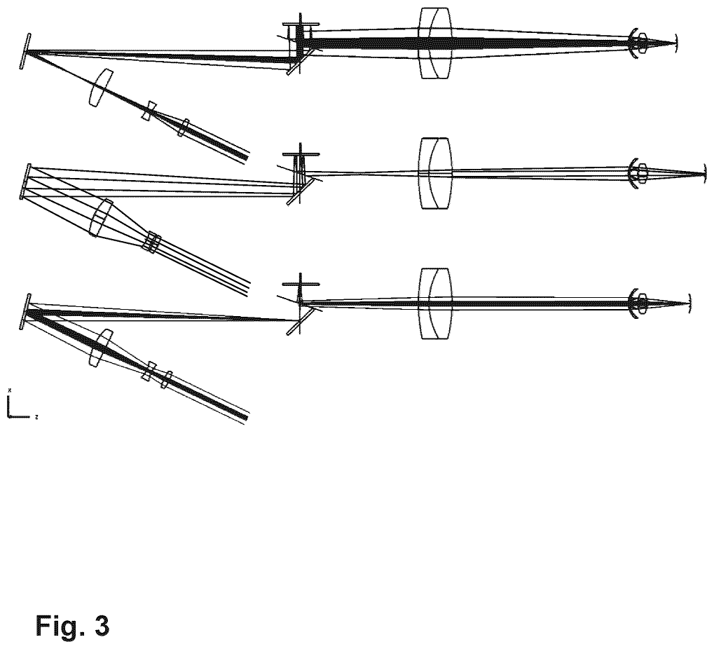

FIG. 3 shows various lens configurations for focusing purposes.

FIG. 1 shows a device 1 with an interferometer. The device 1 comprises a light source 2 and a beam splitter 3, which splits the light coming from the light source 2 into a sample beam on a sample arm 4 and a reference beam on a reference arm 5.

A returning sample beam as returning light 4a is reflected back off a sample 6, specifically an eye, and interferes with a returning reference beam as returning light 5a that is reflected by a mirror 7.

An evaluation unit 8 evaluates the signals of the interfering beams or lights 4a, 5a and creates depth slice images from the signals.

To create the depth slice images, the sample beam is steered to different lateral positions on the sample 6 by a deflection unit 9. These positions define the measurement region 6a.

If need be, an optical unit 10 can focus the sample beam at a certain depth in the sample 6.

The depth slice image is recorded in the depth 11 of the sample 6. The depth 11 can be defined independently of the position or movement of the mirror 7 and independently of the distance 12 between the device 1 and the sample 6 by way of a path length switching unit 13a and/or 13b, which is arranged in the beam path of the sample arm 4 and/or of the reference arm 5.

In this respect, FIG. 1 shows a device 1 for determining the length of an object 6 when performing optical coherence tomography, comprising an interferometer having a light source 2, a sample arm 4 and a reference arm 5, wherein the light transmitted by the light source 2 is splitable by a beam splitter 3 such that first light 4a is guidable on the sample arm 4 in outward and return directions and second light 5a is guidable on the reference arm 5 in outward and return directions, wherein the first and the second returning light 4a, 5a can be made to interfere.

The evaluation unit 8 is arranged for acquiring and processing signals of the interfering first and second lights 4a, 5a, wherein a path length switching unit 13a, 13b is arranged in the beam path of the sample arm 4 and/or in the beam path of the reference arm 5 and modifies the optical path length of the light 4a, 5a passing through the path length switching unit 13a, 13b in each case.

At least one of the path length switching units 13a, 13b changes an optical path length from a first value to a second value alternately in time.

At least one of the path length switching units 13a, 13b changes the first value to the second value after a defined time interval and subsequently, following the expiry of the time interval or a further time interval, changes the second value back to the first value.

The path length switching unit 13a, 13b performs the alternating change of values over a predeterminable time period, preferably in periodic fashion at a defined frequency. The frequency lies in the range of 1 to 1000 Hz.

The difference between the optical path lengths at the first value and at the second value is variably adjustable on the basis of the length of the examined object 6 to be measured, wherein the expected length can be input into the evaluation unit 8 or into a control unit 8a as an external parameter.

Structures of the object are ascertainable by the evaluation unit 8 from the acquired and processed signals, the structures being in each case detectable while the first value and the second value are set.

The spatial distance between these structures is ascertainable and able to be output by the evaluation unit 8 as the length of the object 6.

An adjustable telescope that serves to keep in focus a structure to be captured is provided as optical unit 10.

The evaluation unit 8 differentiates the conjugate complex plane of a signal from the real plane of the signal. A respective numerical phase correction is performable using the evaluation unit 8 in order to ascertain the plane in which a signal of a structure of the object 6 to be captured is located.

A dispersive element, preferably a light-guiding fiber, could be arranged in the reference arm 5 or in the sample arm 4. However, this is not depicted here.

FIG. 2 schematically shows a method for determining the length of an eye, wherein use is made of a device 1 of the above-described type, with at least one path length switching unit 13a, 13b changing an optical path length from a first value to a second value alternately in time in order to bring or focus light into the region of the cornea 14 at the first value and in order to bring or focus light into the region of the retina 15 at the second value.

In FIG. 2, the left-hand column schematically shows setting of first values for detecting the region of the cornea 14, and the right-hand column schematically shows setting of second values for detecting the region of the retina 15. The arrow represents the difference in optical path lengths at the respective values, i.e. the path length offset.

The local position of the cornea 14 is detected at the first value, and the local position of the retina 15 is detected at the second value, with the length of the eye being ascertained from the distance of the positions from one another.

The movement of the eye during the measurements can be detected by evaluating a plurality of measurements, or a series of measurements, of the positions of cornea 14 and retina 15, and the results of this evaluation can be used to correct errors when detecting the cornea 14 and the retina 15.

The retina 15 or its associated signal could be situated either in a real plane or a real image component of an OCT image or in a complex conjugate plane or a complex conjugate image component of the OCT image, with either the movement trajectories of cornea 14 and retina 15 being evaluated for determining the length of the eye or with a phase analysis of the signals from the cornea 14 and retina 15 being performed.

Images of the cornea and of the retina are displayed on a monitor 16 in real time.

Specifically, the eye length is measured as follows using the device 1:

Two optical paths are realized by at least one path length switching unit 13a, 13b.

The first optical path, represented by the first value, has an optical path length just in front of the cornea 14 of the patient that is the same as the path length of the reference arm 5 (“DC position”). It is realized by suitable lenses in such a way that the focus is situated in the region of the cornea 14.

The second optical path, represented by the second value, has an optical path length that is such that the DC position is in the region of the retina 15 of an eye of typical length.

In FIG. 2, the OCT imaging region is indicated by the boxes, the central separating line depicted using dashes in part corresponds to the DC position, the minus sign indicates the conjugate complex plane, and the plus sign indicates the real plane.

The upper boxes show the conditions for a standard eye, the middle boxes show the conditions for a short eye, and the lower boxes show the conditions for a long eye.

The offset depicted by the double-headed arrow is the difference in the path lengths of the two beam paths which is predetermined, in particular predetermined mechanically, by the setup of the path length switching unit 13a, 13b.

A fast switch-over and an alternate measurement of the positions of cornea 14 and retina 15 is required in order to minimize the influence of patient movements on the measurement result.

This is rendered possible in the order of milliseconds by the path length switching unit 13a, 13b. Ideally, an entire series of alternating positions is measured. The movement trajectory of the eye/measurement instrument can be inferred from this series, in order to correct a possible residual error.

During use of the device 1 described here, it is possible for the retina 15 to be situated either in the conjugate complex image component or in the real image component.

An assignment, and hence correct determination of the eye length, can be realized in two ways.

One option lies in checking the movement trajectories between cornea 14 and retina 15, with these being in the same sense if the retina 15 is also in the real image component and these being in the opposite sense if said retina is in the conjugate complex image component, and another is based on a phase analysis of the signals.

To further optimize the signal yield on the retina 15, an optimal focus on the retina 15 can be realized for different eye lengths with the aid of a focus telescope.

To this end, it is possible to realize a lens configuration which achieves a constant degree of fill for the pupil, and so work is always carried out with maximum numerical aperture for the different eye lengths in order to further optimize the SNR. Naturally, this yields higher SNRs for shorter eyes.

FIG. 3 shows such lens configurations by way of example.

List of reference signs:
1 Device
2 Light source
3 Beam splitter
4 Sample arm
4a Outward and return light
5 Reference arm
5a Outward and return light
6 Sample (eye)
6a Measurement region
7 Mirror
8 Evaluation unit
8a Control unit
9 Deflection unit
10 Optical unit
11 Depth
12 Distance between device 1 and sample 6
13a, 13b Path length switching unit
14 Cornea
15 Retina
16 Monitor

1. A device for determining the length of an object when performing optical coherence tomography, comprising an interferometer having a light source, a sample arm and a reference arm, wherein the light transmitted by the light source is splitable by a beam splitter or circulator such that first light is guidable on the sample arm in outward and return directions and second light is guidable on the reference arm in outward and return directions, wherein the first and the second returning light can be made to interfere, wherein an evaluation unit is arranged for acquiring and processing signals of the interfering first and second lights and wherein a path length switching unit is arranged in the beam path of the sample arm and/or in the beam path of the reference arm and modifies the optical path length of the light passing through the path length switching unit in each case, characterized in that at least one path length switching unit changes an optical path length from a first value to a second value alternately in time.

2. The device as claimed in claim 1, wherein the path length switching unit changes the first value to the second value after a defined time interval and subsequently, following the expiry of the time interval or a further time interval, changes the second value back to the first value.

3. The device as claimed in claim 1, wherein the path length switching unit repeats the alternating change of values over a predeterminable time period, preferably in periodic fashion at a defined frequency.

4. The device as claimed in claim 3, wherein the frequency is in the range of 1 to 1000 Hz.

5. The device as claimed in claim 1, wherein the difference between the optical path lengths at the first value and at the second value is variably adjustable on the basis of the length of the examined object to be measured, wherein the expected length can be input into the evaluation unit or into a control unit as an external parameter.

6. The device as claimed in claim 5, wherein structures of the object are ascertainable by the evaluation unit from the acquired and processed signals, the structures being in each case detectable while the first value and the second value are set, and wherein the spatial distance between these structures is ascertainable and able to be output by the evaluation unit as the length of the object.

7. The device as claimed in claim 1, wherein provision is made for an adjustable telescope that serves to keep in focus a structure to be captured.

8. The device as claimed in claim 1, wherein the evaluation unit differentiates the conjugate complex plane of a signal from the real plane of the signal.

9. The device as claimed in claim 8, wherein a respective numerical phase correction is performable using the evaluation unit in order to ascertain the plane in which a signal of a structure of the object to be captured is located.

10. The device as claimed in claim 1, wherein a dispersive element, preferably a light-guiding fiber, is arranged in the reference arm or in the sample arm.

11. A method for determining the length of an eye, wherein use is made of a device as claimed in claim 1, with at least one path length switching unit changing an optical path length from a first value to a second value alternately in time in order to bring or focus light into the region of the cornea at the first value and in order to bring or focus light into the region of the retina at the second value.

12. The method as claimed in claim 11, wherein the local position of the cornea is detected at the first value and wherein the local position of the retina is detected at the second value, with the length of the eye being ascertained from the distance of the positions from one another.

13. The method as claimed in claim 12, wherein the movement of the eye during the measurements is detected by evaluating a plurality of measurements, or a series of measurements, of the positions of cornea and retina and wherein the results of this evaluation are used to correct errors when detecting the cornea and the retina.

14. The method as claimed in claim 11, wherein the retina or its associated signal is situated either in a real plane or a real image component of an OCT image or in a complex conjugate plane or a complex conjugate image component of an OCT image, with either the movement trajectories of cornea and retina being evaluated for determining the length of the eye or with a phase analysis of the signals from the cornea and retina being performed.

15. The method as claimed in claim 11, wherein images of the cornea and the retina are displayed and/or displayed on a monitor in real time.
